(19) United States
(12) Patent Application Publication
(10) Pub. No.: US 2004/0191760 A1
(43) Pub. Date: Sep. 30, 2004

(54) Lateral flow rapid immunoassay test device

(76) Inventors: David F. Zhou, Poway, CA (US); Nai Shu Wang, San Diego, CA (US)

Correspondence Address: CHARMASSON & BUCHACA & LEACH LLP, 1545 HOTEL CIRCLE SOUTH, SUITE 150, SAN DIEGO, CA, 92108-3412, US

(21) Appl. No.: 10/397,121

(22) Filed: Mar. 25, 2003

(51) Int. Cl. C12Q 1/70; G01N 33/554; G01N 33/569; G01N 33/558

(52) U.S. Cl. 435/5; 435/7.32

(57) ABSTRACT

A lateral flow chromatographic immunoassay rapid test device uses a strip having multiple epitope antigen test lines that are responsive to the same pathogenic organism or pathogen. In the preferred embodiment of the invention, a rapid test strip for detection of antibodies to HIV-1, HIV-1/subgroup O, and HIV-2 has test lines coated with P-24, gp41, gp120 , gp160 and gp36 antigens.

### FIELD OF THE INVENTION

This invention relates to chromatographic immunoassay test devices and particularly to lateral flow rapid test devices that group a plurality of test lines on a single strip or multiple strips in a single cassette for detecting various epitopes or antigenic determinants of an exogenous, infectious pathogen or pathogenic organism.

### BACKGROUND OF THE INVENTION

Lateral flow tests based on the principles of chromatographic immunoassay uses strips coated with antigens or antibodies that, upon reaction with antibodies or antigens present in a contacting blood specimen or other sample, result in the appearance of colored lines indicative of the presence in a patient of the pathogenic organism that triggered the formation of the antigens or antibodies. The general format of the strip exhibit a single test line, so-called “T-line” and a single control line, so-called “C-line”. The control line is used as an indicator of functional validity. More recent test strips are offered that group multiple test lines for the detection of more than one kind of substance in the contacted sample. The most common type of multiple test line strips is used for the detection of chemical drugs in blood or urine samples as well as for the detection of indicators of an endogeneous physical disorder such as acute myocardial infarction. No multi-line lateral flow rapid test has been offered for the detection a single exogenous infection in which a plurality of different antigen/antibody complex components responsive to that infection appear on the same strip. Yet, the chance of detection is greatly improved when more than one inter-reactive agent is used.

The present invention results from an attempt to improve the efficacy of lateral flow rapid immunoassay test devices, particularly as a convenient tool in the fight against human immunodeficiency virus (HIV) or hepatitis C virus (HCV).

### SUMMARY OF THE INVENTION

The principal and secondary objects of this invention are to provide a more efficient and more discriminative way of rapidly detecting HIV, HCV and other infections through the use of lateral flow chromatographic immunoassay test devices and thus, avoid the long turn-around time required by use of separate assays using multiple tests such as in a Western Blot assay.

These and other valuable objects are achieved by grouping on a single test strip, lines coated with a plurality of immunoassay complex components that although different, are all responsive to the same pathogenic organism. Each immunoassay complex components may be an antigen whose epitope component will inter-react with an antibody paratope in the sample, or an antibody whose paratope component will inter-react with an antigen epitope. This novel rapid test format can also be used for multiple pathogenic organisms with multiple epitope test lines in one single cassette (plastic housing).

### BRIEF DESCRIPTION OF THE DRAWING

FIG. 1 is a diagrammatical illustration of a multi-line test strip according to the invention;

FIG. 2 is a diagrammatical illustration of a chromatographic immunoassay test device;

FIG. 3 is a diagrammatical illustration of a second type of a multi-line test strip;

FIG. 4 is a diagrammatical illustration of first type of multi-strip test device; and

FIG. 5 is a diagrammatical illustration of a second type of multi-strip test device.

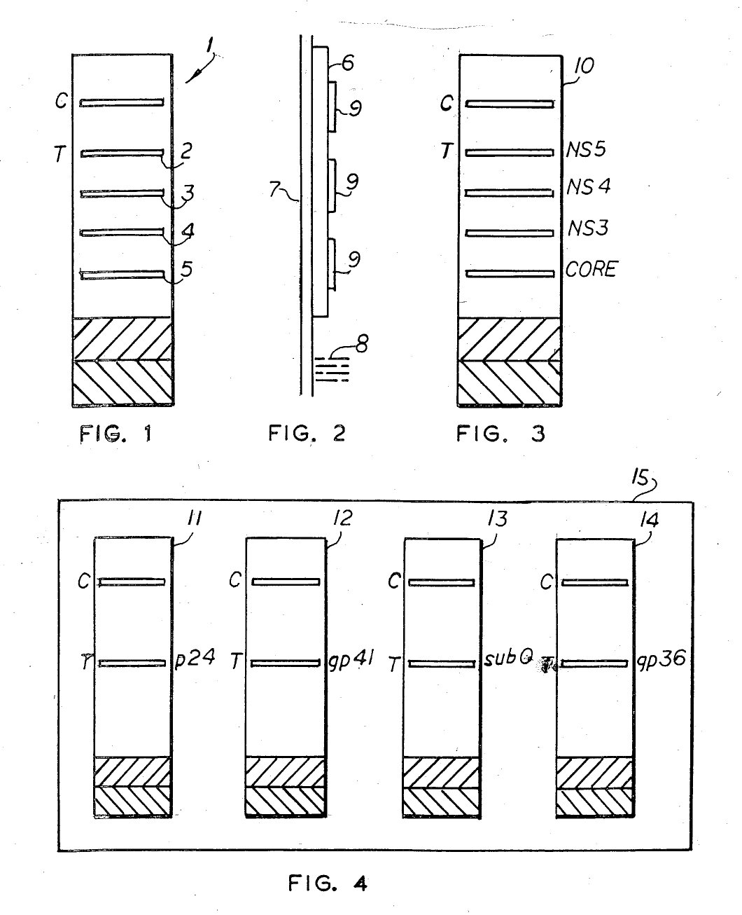

### DESCRIPTION OF THE PREFERRED EMBODIMENT OF THE INVENTION

Referring now to the drawing in a first example, a test strip 1 adapted for use on a lateral flow rapid chromatographic immunoassay test device comprises a plurality of test lines 2, 3, 4 and 5 coated with high sensitivity and specificity epitope antigens that are all responsive to various forms of HIV. More specifically, a first test line 2 is coated with gpi60 antigen, a second line 3 is coated with sub o antigen, a third line 4 is coated with gp41 antigen, and a fourth line 5 is coated with p24 antigen. Such a test strip can be effectively used to detect the presence of antibodies triggered by HIV-1, HIV-1/subgroup O, and HIV-2 in a body fluid sample. The antigen gp120 could also be used in addition or in lieu of the antigen gp36.

As more specifically illustrated in FIG. 2, each line on the strip comprises a nitrocellulose membrane 6 coated with the antigens. The membrane is in contact with a conjugate release pad 7 containing colloidal gold-conjugated HIV antigens that capture the HIV specific antibodies present in the sample 8 by which it is contacted. As the HIV antibody-antigen complexes pass through the conjugated pad by capillarity action, they are captured by the unlabeled HIV antigens 9 coated on the nitrocellulose membranes, each on a different line. Each specific biochemical interaction causes the appearance of a burgundy-colored test line.

No test line indicates that the antibody concentration is below the level of detection, or is absent altogether. This novel format gives a more conclusive result than a single-line HIV rapid, lateral flow test.

Particularly remarkable results were obtained using Boston Biomedica 25-member Anti-HIV-1 Mixed Titer Performance Panel (PRB 203, 15-member Anti-HIV-1 Low Titer Performance Panel (PRB 107), 3-member HIV-1 Seroconversion Panel (PRF 202) and 15-member Anti-HIV-1/2 Combo Performance Panel.

In a second example illustrated in FIG. 3, a multii-test line strip 10 is formulated for the detection of antibodies to the hepatitis C virus (HCV) using antigens NS3, NS4, NS5 and core.

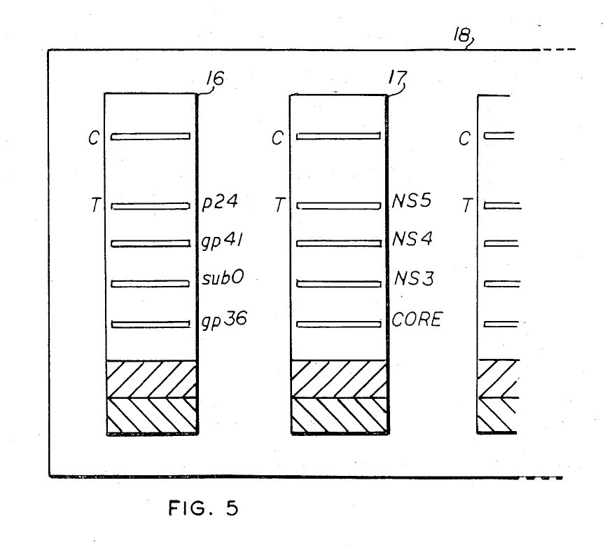

In a third example illustrated in FIG. 4, four strips 11, 12, 13, 14 are combined in a panel format under a single plastic housing 15 wherein each strip carries a distinct antigen responsive to an antibody triggered by the distinctive epitope of the same exogenous pathology as the other strips, namely, the same various epitope antigens as in the first example. It should be understood that two or more single or multi-line strips can be combined in a single housing cassette for the detection of several exogenous pathogens. As illustrated in FIG. 5, strips 16 and 17 are intended to react to two different pathogenic organisms, but are grouped under the same housing 18. The first strip 16 mounts lines coated with immunoassay complex components that are responsive to HIV while the second strip 17 features test lines addressed to the detection of HCV.

A variety of the above-described test device can be made to detect multiple major epitopes, i.e., antigenic determinants of a single exogenous pathogen, or pathogenic organism such as bacteria, virus, parasites, rickettsia, etc. . . .

While the preferred embodiment of the invention has been described, modifications can be made and other embodiments may be devised without departing from the spirit of the invention and the scope of the appended claims.

What is claimed is:

1. A lateral flow chromatographic immunoassay rapid test apparatus which comprises multiple immunoassay complex component test lines, said lines being coated with a plurality of different epitope immunoassay complex components;
wherein all of said plurality of components are responsive to inter-reactive complex components triggered by a same exogenous infectious pathogenic organism or pathogen.

2. The apparatus of claim 1 which further comprises at least one strip inclusing at least one nitrocellulose membrane mounting said test lines.

3. The apparatus of claim 2 which further comprises a conjugate release pad in contact with said strip;
said pad being formulated to capture said inter-reactive complex component in a contacting sample.

4. The apparatus of claim 1, wherein said epitope components are antigens taken from a group consisting essentially of p24, gp41, sub O, gp36, gp120, and gp160.

5. The apparatus of claim 4, wherein said pathogenic organism comprises a form of HIV.

6. The apparatus of claim 1, wherein said epitope components are antigens taken from a group consisting essentially of NS3, NS4, NS5 and core; and
said pathogenic organism consists of HCV.

7. The apparatus of claim 1 which further comprises a single housing containing a strip mounting all of said epitope components, each on a separate line.

8. The apparatus of claim 1 which further comprises a single housing containing a plurality of strips, each of said strips mounting at least one of said epitope components.

9. The apparatus of claim 7 which further comprises at least one additional strip in said housing;
said additional strip having multiple test lines coated with a set of different immunoassay complext components;
wherein all of said sets of components are responsive to inter-reactive complex components triggered by an exogenous infectious pathogenic organism or pathogen other than said same exogenous infectious pathogenic organism or pathogen.

10. A method for detecting an exgenous infectious pathogen by means of a lateral flow chromatographic immunoassay rapid test on a sample of a patient's body fluid which comprises:
exposing said sample to a strip having a plurality of test lines each of said test lines carrying an epitope immunoassay complex component responsive to at least one inter-reactive component indicative of the presence of said pathogen in said patient;
wherein each of said test lines carries an epitope component different from epitope components on any other of said test lines.

11. The method of claim 10, wherein said exgenous infectious pathogen comprises HIV, and said each of said epitope component is an antigen selected from a group consisting essentially of p24, gp36, gp41, sub0, gp120 and gp160.

12. The method of claim 10, wherein said exgenous infectious pathogen consists of HCV, and said each of said epitope complex is an antigen selected from a group consisting essentially of NS3, NS4, NS5 and core.
